(19) United States
(12) Patent Application Publication
(10) Pub. No.: US 2024/0044919 A1
(43) Pub. Date: Feb. 8, 2024

(54) SYSTEM, METHOD AND DEVICE FOR DETECTING AND MONITORING POLYCYSTIC OVARY SYNDROME

(71) Applicant: Varun AKUR VENKATESAN, Bangalore (IN)

(72) Inventors: VARUN AKUR VENKATESAN, BANGALORE (IN); SIDDARTH PATTNAIK, BANGALORE (IN); DIPANKAR DAS, BANGALORE (IN)

(21) Appl. No.: 18/266,243

(22) PCT Filed: Dec. 8, 2021
(86) PCT No.: PCT/IN2021/051148

§ 371 Date: Jun. 8, 2023

(30) Foreign Application Priority Data
Dec. 8, 2021 (IN) 202011024021

(51) Int. Cl. G01N 33/74; G16H 50/20

(52) U.S. Cl. CPC G01N 33/743; G16H 50/20; G01N 2333/59; G01N 2800/367

(57) ABSTRACT

A method and handheld computer-implemented system for diagnosing or predicting poly-cystic ovarian syndrome by measuring reproductive hormones or their metabolites in a biological sample of a subject is provided.

### FIELD OF INVENTION

The present embodiment relates to devices, systems and methods to detect or predict diseases/disorders; and more particularly to detecting and monitoring polycystic ovary syndrome (PCOS).

### SUMMARY OF THE PRIOR ART

There is no single test to predict, detect or establish Polycystic ovary syndrome (PCOS) in a patient. Usually, medical professionals ask about symptoms, conduct a number of blood tests and physical exam before deciding whether a patient is suffering from PCOS. PCOS is a hormone disorder that causes problem with fertility, weight, hair, menstruation and skin. Usually, PCOS is diagnosed on basis of symptoms, especially if two of the three symptoms [Rotterdam criteria] i.e. irregular period, high levels of male hormones through variety of tests or by symptoms and presence of cysts in ovaries [determined by X-ray exam], are shown by a patient, a PCOS diagnosis is confirmed.

A number of blood tests are conducted on the blood sample drawn from vein to measure levels of Follicle-stimulating hormone (FSH), Luteinizing hormone (LH), Testosterone, Estrogens, Human chorionic gonadotropin (hCG) and Anti-Mullerian hormone (AMH) to arrive at diagnosis of PCOS together with physical and X-ray exam.

With poly-cystic ovarian syndrome (PCOS) being the most common cause of infertility, occurring in approximately 1 in every 5 women, and with 70% women (per one estimate) being undiagnosed of PCOS since it does not present any apparent symptoms, a personal, home-based screening tool or point-of-care system may help women in being proactive in detecting/predicting PCOS in time, and approach their healthcare provider to get a confirmatory test, and then subsequently use the point-of-care or home-based screening tool to manage PCOS to improve fertility, and prevent related diseases such as metabolic and cardiovascular diseases. Furthermore, PCOS is usually managed by diets and exercise, and therefore requires more dynamic prediction or diagnosis of PCOS so that the effect of exercise and diet on PCOS levels in a subject may be monitored. Currently diagnostic methodologies do not allow such dynamic and regular prediction or scoring of PCOS.

In view of the foregoing, there is a need to develop a handheld device, system and method that allows screening or detecting or predicting of PCOS at the point of care, especially a home-based, personal and handy system, method and device for screening, detecting or predicting and monitoring PCOS.

### SUMMARY OF THE INVENTION

As mentioned in the foregoing, the embodiment herein provides a method of screening and monitoring polycystic ovary syndrome (PCOS) in a patient by simultaneously measuring and monitoring hormones using a handheld device.

In an aspect, a method for diagnosis and monitoring of poly-cystic ovarian syndrome in a female subject by analysing a biological sample taken from the subject is provided. The method includes quantifying concentration or level of one or more reproductive hormones in free or bound form. The one or more reproductive hormones or their metabolites are selected from the list consisting of E3G, LH, PDG, FSH, DHEA and Testosterone in free or bound form. The method as claimed in claim 1 further includes measuring the levels of said reproductive hormones or their metabolites during at least one menstrual cycle; determining a maximum concentration of said reproductive hormones or their metabolites during at least one menstrual cycle; determining simultaneously, for the same cycle, for one or more of said hormones whether the concentration of one or more of said hormones is greater than or lesser than a threshold value during follicular phase of said cycle.

In another aspect, a method for diagnosis of poly-cystic ovarian syndrome in a female subject by analysing a biological sample taken from the subject is provided. The method includes quantifying concentration/level of luteinizing hormone (LH) and of an Estrogen metabolite such as Eestrone-3-glucuronide (E3G) in the sample during at least one cycle; determining a maximum concentration/level LHₘₐₓ of LH and a maximum concentration/level Eₘₐₓ of E3G (or any Estrogen metabolite) during at least one menstrual cycle; determining, simultaneously, for same cycle, whether the Estrogen metabolite levels in the sample are more than 0.8 times Eₘₐₓ during a follicular phase of the cycle and whether the LH levels are less than 0.095 times LHₘₐₓ during follicular phase of the cycle.

The method further includes placing the biological sample over a sample receiving well/zone of a test strip; reacting the treated biological sample with a detector reagent (a conjugate) containing a detector antibody, which conjugates with the LH and aby of the Estrogen metabolites, e.g., E3G in the biological sample and flows with the biological sample; receiving of the biological sample fluid along with the detector antibody on at least one test and control lines having immobilized biochemical reagent leading to a coloured reaction giving at least one characteristic colour; and quantification of E3G (or any other chosen Estrogen metabolite) and LH in the biological sample by comparing the characteristic colours against control. In the method, the biological sample is urine. The method further includes scanning the immunochromatographic assay strips by a handheld device having an imaging sensor for quantification of any Estrogen metabolite and LH in the sample by comparing the colour on the strips against a standard or control.

In another aspect, a method for diagnosing poly-cystic ovarian syndrome (PCOS) in a female subject by analysing a urine sample in the female subject is provided. The method includes measuring levels of luteinizing hormone (LH) and estrone-3-glucuronide (E3G) or any Estrogen metabolite in a biological sample during follicular phase of a menstrual cycle; determining whether E3G levels in the biological sample are more than 17 ng/mL along with whether LH levels are less than 2.4 mIU/mL [0.020 ng/ml] during follicular phase of the menstrual cycle.

The method further includes placing the urine sample over a sample receiving pad of a test strip, that pre-treats the biological sample; reacting the treated urine sample with a coloured detector reagent (a conjugate) containing a detector antibody, which is re-hydrated and flows with the urine sample through a membrane of the test strip, receiving of the urine sample fluid along with the detector antibody on at least one test and control lines having immobilized biochemical reagent leading to a coloured reaction giving at least one characteristic colour; and quantification of E3G and LH in the urine sample by comparing the characteristic colours against control.

In yet another aspect, a handheld computer-implemented system for diagnosing or predicting poly-cystic ovarian syndrome by measuring E3G or any Estrogen metabolite and LH levels in a biological sample of a subject is provided. The system includes plurality of test strips (202, 204 . . . 20N) to receive the biological sample, wherein the test strips are immunochromatographic assay strips to detect E3G and LH in the sample; an imaging sensor (204) to scan the test strips (202, 204, 20N); an E3G (or any Estrogen metabolite) quantification module (206) that is in communication with the imaging sensor (204) and receives scanned images from the imaging sensor (204) to quantify the immunochromatographic assay by comparing the colour on the strips against a standard or control; an LH quantification module (208) that is in communication with the imaging sensor (204) and receives scanned images from the imaging sensor (204) to quantify the immunochromatographic assay strips by comparing the colour on the strips against a standard or control; an analytics module (210) that is in communication with the E3G quantification module (206) and the LH quantification module (208) for plotting quantification data from the E3G quantification module (206) and the LH quantification module (208); and a diagnostics module (212) that is in communication with the analytics module (210). The diagnostics module (212) determines if E3G levels (or any Estrogen metabolite) are more than LH levels in the sample for at least a plurality of cycle days, The handheld device has the imaging sensor. The handheld device hosts the E3G and the LH quantification modules (206 and 208), the diagnostics module (210), the analytics module (212) and a display module (214). The diagnostics module (212) determines whether E3G levels in the urine sample is more than 17 ng/mL along with LH levels of less than 2.4 mIU/mL [0.020 ng/ml] during follicular phase of the menstrual cycle. The E3G quantification module (206), the LH quantification module (208), the analytics module (210) and the diagnostics module (212) are hosted on a remote server that is communication with the handheld device (106). The LH and E3G immunochromatographic assays are performed on a single strip.

These and other aspects of the embodiments herein will be better appreciated and understood when considered in conjunction with the following description and the accompanying drawings. It should be understood, however, that the following descriptions, while indicating preferred embodiments and numerous specific details thereof, are given by way of illustration and not of limitation. Many changes and modifications may be made within the scope of the embodiments herein without departing from the concept thereof, and the embodiments herein include all such modifications.

### BRIEF DESCRIPTION OF THE DRAWINGS

Other objects, features, and advantages of the embodiment will be apparent from the following description when read with reference to the accompanying drawings. In the drawings, wherein like reference numerals denote corresponding parts throughout the several views:

FIG. 1 illustrates handheld imaging system to measure E3G and LH hormones in a subject, according to an embodiment herein;

FIG. 2 illustrates an embedded computer-implemented system for screening polycystic ovaries syndrome (PCOS) using handheld system of FIG. 1, according to an embodiment herein;

FIG. 3 illustrates a plot prepared by the analytics module 210 of the system of FIG. 2, according to an embodiment herein;

FIG. 4 illustrates a method of diagnosing PCOS by measuring E3G and LG levels in a biological sample, according to an embodiment herein; and

FIG. 5 illustrates a method of monitoring PCOS in a subject, according to an embodiment herein.

### DETAILED DESCRIPTION OF THE PREFERRED EMBODIMENTS

The embodiments herein and the various features and advantageous details thereof are explained more fully with reference to the non-limiting embodiments that are illustrated in the accompanying drawings and detailed in the following description. Descriptions of well-known components and processing techniques are omitted so as to not unnecessarily obscure the embodiments herein. The examples used herein are intended merely to facilitate an understanding of ways in which the embodiments herein may be practiced and to further enable those of skill in the art to practice the embodiments herein. Accordingly, the examples should not be construed as limiting the scope of the embodiments herein.

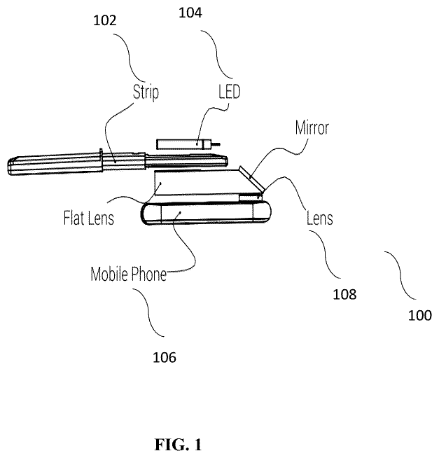

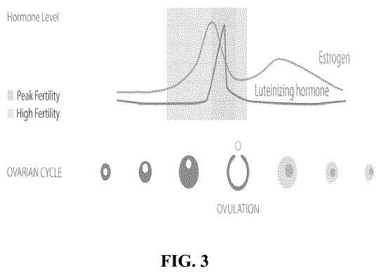

As mentioned, there remains a need for handheld devices, especially to enable personal, home-based and/or point-of-care screening, diagnosis, predicting, or detecting polycystic ovary syndrome (PCOS). Referring now to the drawings, and more particularly to FIG. 1 through FIG. 4, where similar reference characters denote corresponding features consistently throughout the figures, there are shown several embodiments of a device, system and method to detect, screen, diagnose, predict PCOS in a subject based on a biological sample from a subject after quantifying quantities of one or more reproductive hormones or their metabolites. In an embodiment, the reproductive hormones are selected from Eesterone-3-glucuronide (E3G), Luteinizing Hormone (LH), Pregnanediol Glucuronide (PDG), Follicle Stimulating Hormone (FSH), Dehydroepiandrosterone (DHEA) and Testosterone in free or bound form. In a preferred embodiment, the hormones to be detected for diagnosing or predicting PCOS includes Luteinizing Hormone as well as any Estrogen metabolite. In a preferred embodiment, Eestrone-3-glucuronide (E3G) is the estrogen metabolite. In the present disclosure, Eestrone-3-glucuronide (E3G) is taken as an example or representative example of an Estrogen metabolite. The term “handheld device” here refers to any device that is capable of reading a strip that receives a biological sample, and reacts with reagents [loaded on strip] and detects a particular or desired analyte's presence or absence or levels in the biological sample. The handheld device may be a smartphone, a stand-alone system or a dedicated metre or device to read such strips.

The term “biological sample” refers to any bodily fluid or fluid type substance from a subject/patients to determine the presence, absence or levels of any hormones, metabolites, minerals, etc. in the subject's body. The biological sample may include sweat, urine, blood, blood serum, semen, breast milk, saliva, blood plasma, tears, mucus, cerebrospinal fluids, saliva, amniotic fluid, vaginal lubrication fluids, pus, lymph, bile, synovial fluid, aqueous humour, phlegm, gastric acid, pre-ejaculate, colostrum and other such fluids related to humans.

The term “test strip” refers to a medium or base or any paper-based device on which a biochemical reagent is affixed or immobilized to react with an analyte [present in the biological sample taken from the subject] to determine presence, or absence and measure the amount of the analyte in the subject. The test strip may be immunoassay strips, which, in turn, may be immunochromatographic strips. The test strip or assay may be a lateral flow assay.

The term “cycle” or “menstrual cycle” refers to a cycle of menstruation in a female subject that is counted from the first day of 1 period to the first day of the next period. The average menstrual cycle is 28 days long. In an embodiment, the cycles range from 21 to 35 days in adults and from 21 to 45 days in young teens.

The term “follicular phase” of the menstrual cycle refers the proliferative phase when follicles in the ovary mature, and which ends with ovulation.

The term “luteal phase” of the menstrual cycle is the phase that starts after the ovulation and lasts until first day of the menstruation.

FIG. 1 illustrates a handheld imaging system (100) to measure E3G and LH hormones in a subject, according to an embodiment herein. The handheld imaging system (100) includes a strip reader (102), a lighting means (104), a handheld device (106) and an optical waveguide (108). The handheld device (106) captures an image of the strip reader (102) carrying a test strip, which is illuminated by the lighting means (104). In a preferred embodiment, the optical waveguide (108) is included in the imaging system so as to steer beam from the lighting means in desired direction i.e. towards mirror, which directs incoming light to a camera of the handheld device (106). In an embodiment, the optical waveguide includes a transparent optical block, a thin layer of highly reflective prism [that steers the beams], mirror to direct the incoming light to the camera, and a plano-convex lens that couples the camera of the handheld device with the optical wave guide.

As mentioned in the foregoing, the test strips, which are read by the strip reader (102) may be immunochromatographic strips, and more particularly, lateral flow assay strips.

In an embodiment, the immunochromatographic strip is a paper-based lateral flow-assay strip that enables chromatic immunoassays. The test strip includes a porous membrane under which a capturing biochemical reagent is immobilised in different “lines” such as a control line and a test line.

The biological sample, which may be carrying an analyte, is placed over a sample pad of the test strip, which pre-treats the sample before it reacts with a coloured detector reagent (a conjugate immobilised on a conjugate pad), usually colloidal gold, containing a detector antibody, which is re-hydrated and flows with the sample through the strip. The sample fluid along with conjugate antibody travels through porous membrane consisting of test and control lines having immobilized biochemical reagent. The reaction of the conjugated sample fluid with the immobilised biochemical reagent [usually another antibody i.e. leading to a sandwich assay], on the test line, gives a characteristic colour of the reaction that tells the presence an analyte in the biological sample and by comparing the colour intensity on the test line with the control line, the quantification of the analyte is done.

The test strip also includes a sample receiving well/zone that receives the biological sample to be analysed for quantification of the desired analyte. The sample receiving well/zone may contain buffers or surfactants to pre-treat the biological sample before it travels up the lateral flow strip.

As an example, in an embodiment, a urine sample or its extract from a subject and having any of the reproductive hormones or metabolites viz. one or more selected from either Eesterone-3-glucuronide (E3G), Luteinizing Hormone (LH), Pregnanediol Glucuronide (PDG), Follicle Stimulating Hormone (FSH), Dehydroepiandrosterone (DHEA) or Testosterone in free or bound form, are placed on the sample receiving well/zone of the lateral flow assay strip, and the urine sample travels up the conjugate pad having colloidal gold having detector antibodies for Eesterone-3-glucuronide (E3G), Luteinizing Hormone (LH), Pregnanediol Glucuronide (PDG), Follicle Stimulating Hormone (FSH), Dehydroepiandrosterone (DHEA) and Testosterone in free or bound form such that the one or more reproductive hormones in the sample are conjugated to the detector antibodies, and the urine or any biological sample continues to travel up towards the test line and control lines having immobilised secondary antibody or biochemical reagent, upon reaction with which the urine sample having any of the reproductive hormones or their metabolites conjugated with their specific detector antibodies in a gold colloidal gives a characteristic colour on the test line showing the presence of the one or more of the reproductive hormones or their metabolites.

As another example, in a present embodiment, a urine sample [or its extract] from a female subject and having both E3G (or any other Estrogen metabolite) and LH is placed on the sample receiving well/zone of the lateral flow assay strip [which may be mode of a polymer, nitrocellulose or other such material that allow capillary flow of liquids in lateral direction] and the urine sample travels up to the conjugate pad through capillary action to the conjugate pad having colloidal gold having detector LH and E3G (or any other Estrogen metabolite) antibodies, where the urine sample's LH and E3G gets conjugated to the detector LH and E3G antibodies, and the urine sample continues to travel up towards the test line and control lines having immobilised secondary antibody or biochemical reagent, upon reaction with whom the urine sample having LH and E3G conjugated with LH and E3G antibodies in a gold colloidal gives a characteristic colour on the test line showing the presence of the LH and E3G in the urine sample. The quantification of the E3G and LH is then done by measuring the intensity of the colour of the test line. The LH and E3G antibodies may be conjugated to coloured or fluorescent particles

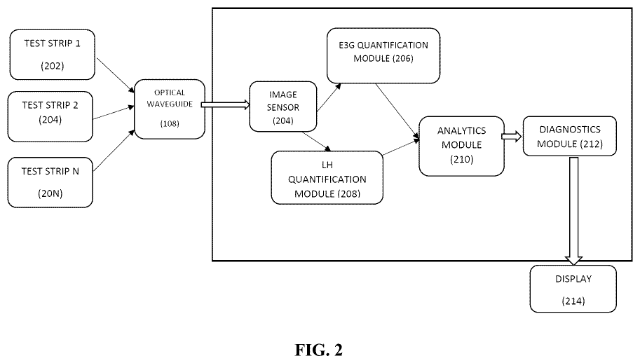

FIG. 2 illustrates an embedded computer-implemented system 200 for screening polycystic ovaries syndrome (PCOS) by using the handheld imaging system 100 of FIG. 1, according to an embodiment herein. The system 200 includes a number of test strips viz, test strip 1 202, test strip 2 204 and test strip n 20N, an optical waveguide 108, an imaging sensor 204, a plurality of reproductive hormone or their metabolites quantification module (206, 208), a computational module 210, a diagnostics module 212, and a display 214. The test strips (201, 202, . . . 20N) are read by the imaging senor 204 via the optical waveguide 204 as described in the foregoing. In an embodiment, and for sake of explanation, the quantification module shown in FIG. 2 represents E3G (or any other Estrogen metabolite) quantification module 206, LH quantification module 208. Other quantification modules that may be added include Pregnanediol Glucuronide (PDG) quantification module, Follicle Stimulating Hormone (FSH) quantification module, Dehydroepiandrosterone (DHEA) quantification module and Testosterone quantification module.

In a preferred embodiment, the imaging sensor 204 is usually and preferably embedded within a handheld device such as a smartphone, whereas the optical waveguide 108 is attached to the imaging sensor 204 via an adaptor. The test strips 202, 204, 20N are scanned by the imaging sensor 204, which is in communication with all of the hormone quantification modules such as E3G quantification module 206 and LH quantification module 208. The handheld device 106 can host the quantification modules such as 206 and 208. Preferably, the quantification modules such as 206 and 208 are hosted on a cloud that is communication with the handheld device 106.

In an embodiment, the scan of the test strips 202, 204, 20N and the quantification of one or more of the reproductive hormones and their metabolites performed at the handheld device 106. In a preferred embodiment, while the handheld device performs the scan of the test strips 202, 204, 20N, the quantification of the hormones is performed at the cloud or a remote server, which is in communication with the handheld device to receive the scanned images of test strips 202, 204, 20N. After the quantification of any of the reproductive hormones or their metabolites, such as E3G and LH by the respective quantification modules 206 and 208, the quantification data of the E3G and LH in the sample, over several cycles, is communicated to the analytics module 210 that further computes the data over several cycles (i.e. menstruation cycles) to determine and compare the E3G levels and LH levels in a subject. In an embodiment, all the quantification modules related to all the reproductive hormones or their metabolites is communicated to the analytics module 210 that computes the data of different hormones over several cycles to determine and compare their levels. As an example, the analytics module 210 analyses the highest level of E3G i.e., Eₘₐₓ and the highest level of LH i.e., LHₘₐₓ over a cycle and the comparisons are communicated to the diagnostics module 212 that predicts/diagnose and/or displays a PCOS diagnosis (i.e., positive or negative) through a display 214 on the handheld device 106. The quantification modules such as 206 and 208 quantify the hormones or their metabolites such as E3G and LH levels in a sample of a subject by quantifying the colour or colour reaction against a standard or control.

The analytics module 210 then plots quantification data of the quantification modules 206 and 208 against days. The diagnostics module 212 determines whether the E3G levels in the sample are more than a first threshold during follicular phase of a menstrual cycle and whether the LH levels are less than a second threshold during same follicular phase. In an embodiment, the first threshold is 0.8 times E. and the second threshold 0.095 times LHₘₐₓ. Similarly, for different reproductive hormones or their metabolites, threshold values are obtained and put in relation with each other. In another embodiment, the follicular phase refers to at least 4 days during 8^{th} to 13^{th} day of a cycle. If two or more conditions are met in a sample on the basis of processing at the diagnostics module communicates a diagnosis of PCOD/PCOS. This relation has been observed to be true for any sample type. However, the preferred biological sample is urine.

In an embodiment, when sample is urine of a female subject, the diagnostics module determines or computes if the E3G levels are more than 17 ng/mL along with LH levels of less than 2.4 mIU/mL [0.020 ng/ml] during follicular phase and if these conditions are satisfied in a urine sample, a positive diagnosis for the PCOS is displayed at the display 214. In an embodiment, the follicular phase refers to at least 4 days during 8^{th} to 13^{th} day of a cycle

In embodiment, a method of developing a machine learning model by including each hormone's level and their correlation with PCOS or related symptoms is included. The continuous feeding of such data to the machine learning model may develop a newer correlation.

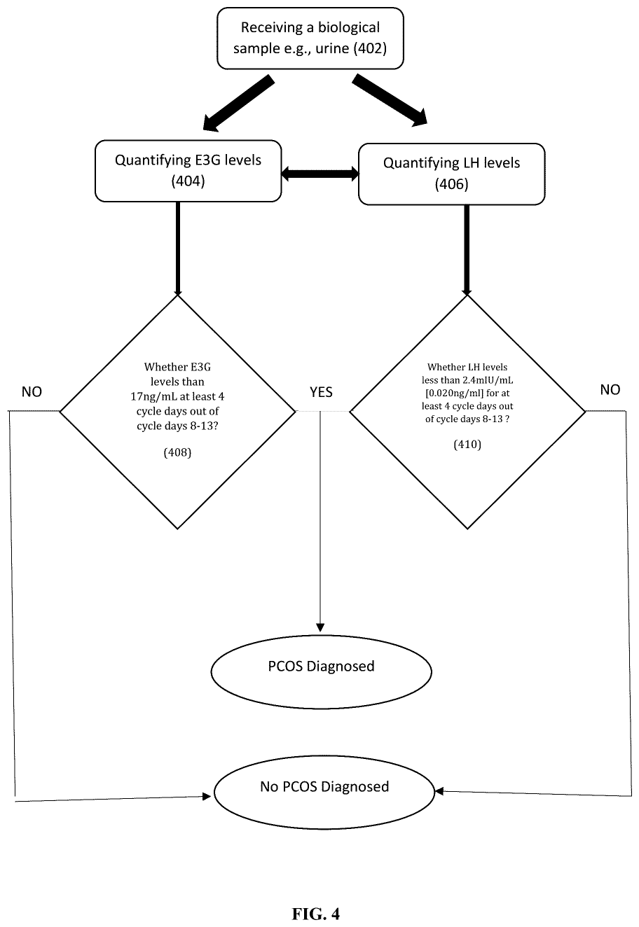

FIG. 4 illustrates a method of diagnosing PCOS by measuring E3G and LG levels in a biological sample, according to an embodiment herein. The method includes receiving (402) a biological sample such as urine or any biological fluids for measuring levels of one or more of the reproductive hormones or their metabolites such as Eesterone-3-glucuronide (E3G), Luteinizing Hormone (LH), Pregnanediol Glucuronide (PDG), Follicle Stimulating Hormone (FSH), Dehydroepiandrosterone (DHEA) and Testosterone in free or bound form. In an embodiment, the concentration or levels of luteinizing hormone (LH) (406) and estrone-3-glucuronide (E3G) (404) in the biological sample are measured. In a preferred embodiment, the levels of LH and E3G in the urine sample is determined during follicular phase. The method further includes determining whether E3G levels (408) in the urine sample is more than 17 ng/mL along with determining whether the LH levels (410) are less than 2.4 mIU/mL [0.020 ng/ml] during follicular phase. If these conditions are met, a PCOS diagnosis is confirmed. In an embodiment, the follicular phase refers to at least 4 days during 8^{th} to 13^{th} day of a cycle

The method further includes placing the biological sample over a sample receiving pad of a test strip, that pre-treats the biological sample; reacting the treated biological sample with a coloured detector reagent (a conjugate) containing a detector antibody, which is re-hydrated and flows with the biological sample through a membrane of the test strip, receiving of the biological sample fluid along with the detector antibody on at least one test and control lines having immobilized biochemical reagent leading to a coloured reaction giving at least one characteristic colour; quantification of E3G and LH (or any of the reproductive hormones or their metabolites) in the biological sample by comparing the characteristic colours against control. In the method, the hormone levels are determined by quantifying immunochromatographic assay strips that receives the biological sample. The immunochromatographic assay strips are scanned by a handheld device having an imaging sensor for quantification of hormones in the sample for quantifying hormone levels in the sample by comparing the colour on the strips against a standard or control.

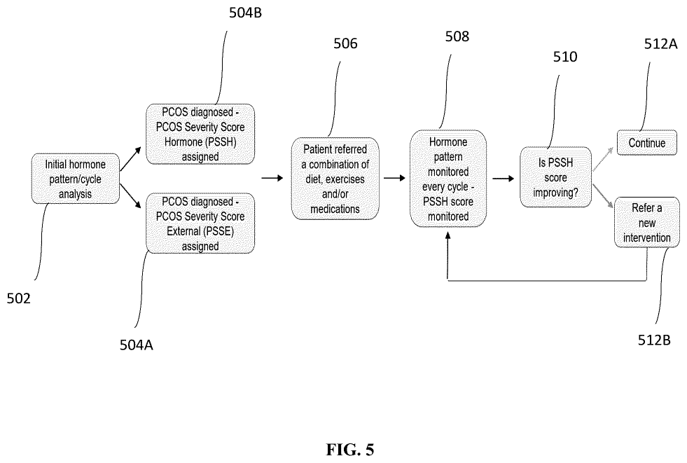

FIG. 5 illustrates a method of monitoring PCOS in a subject, according to an embodiment herein. The method 500 includes analysing, in step 502, patterns of concentration/levels of each of the hormones e.g., Eesterone-3-glucuronide (E3G), Luteinizing Hormone (LH), Pregnanediol Glucuronide (PDG), Follicle Stimulating Hormone (FSH), Dehydroepiandrosterone (DHEA) and Testosterone in free or bound form as discussed in the foregoing. Based on the patterns/concentration of the hormones, a PCOS Severity Score Hormonal (PSSH) is assigned in step 504A. The score may be computed either as weighted means, or median value or an average of concentration/levels of different hormones. In an embodiment, a statistical analysis is run to compute the PCOS severity score. The PCOS Severity Score Hormone refers to the score derived entirely on basis of concentration/levels of hormones in the biological sample as determined by the systems and methods described herein. In Step 504B, a PCOS Severity Score External (PSSE) is computed alongside the step 504A. The PCOS Severity Score External is computed on the basis of external factors or apparent symptoms such as hirsutism of PCOS. The PSSE is computed on basis of the subject's self-declaration of severity of several symptoms and the scoring of each symptom is done by the subject. Based on PSSE and PSSH scores, in step 506 a combination of diet, medications and/or exercises may be displayed or communicated to the subject. Using the method, the PSSH and PSSE are monitored during every cycle and compared with the historical values. In an embodiment, PSSH score improves while PSSE score may worsen or stay same, or vice versa—accordingly, the subject may be referred to see a medical professional or be advises or communicated alternative diet, exercise and/or medication.

In an embodiment, the PSSH score is also assigned based on one or more of the following hormone patterns: FSH levels, LH:FSH ratio, non-ovulatory high LH, absence of coasting type E3G, recurrent oscillation in LH levels, multiple PdG peaks. In an embodiment, the PSSE score is assigned based on one or more of the following cycle parameters/physical characteristics patterns such as frequency of anovulatory cycles, cycle length variation, phenotypic occurrences such as facial hair, acne, baldness or thinning hair, and weight gain. The method discussed in FIG. is used to reevaluate PSSE to confirm the effects of intervention.

### EXAMPLES

### Example 1: Computing PSSH and PSSE Score

The PSSH profile or score of a female subject was determined as 75 on basis of the following:
1. High testosterone (PSSH contribution: 20)
2. High non-coasting estrogen (PSSH contribution: 20)
3. Low FSH, High LH:FSH ratio (PSSH contribution: 15)
4. No PdG rise for 3 continuous cycles (PSSH contribution: 25)

Similarly, the PSSE profile or score of 65 was computed on basis of following:
1. Hirsutism (facial hair) (PSSE contribution: 20)
2. Frequent occurrence of acne (PSSE contribution: 15)
3. Obesity (PSSE contribution: 30)

### Example 2: Diagnosis

Hirsutism is a symptom of increased testosterone concentration in the female subject. The testosterone converts to estrogen due to aromatase in fat cells. Anovulatory cycles could be because of lack of growing follicles.

### Example 3: Intervention

Based on the above scores and diagnosis, the following intervention is communicated to the subject:
1. Letrozole, cycle day 2-6: For inhibiting aromatase and letting the FSH rise
2. Metformin: For obesity
3. Low to medium exercise

### Example 4: Evaluation after 4-5 Cycles

After 4-5 cycles, the subject was revaluated for PSSH and PSSE score as follows:
a. PSSH profile: Total score 42
1. Testosterone levels lowered (PSSH contribution: 10)
2. Coasting observed along with expected rise and drop of estrogen (PSSH contribution: 10)
3. FSH started rising in early part of the cycle (PSSH contribution: 9)
4. PdG rises after LH peak in 2 out of 5 cycles (PSSH contribution: 13)
PSSE profile: Total score 28
1. Lesser facial hair as a result of lower T (PSSE contribution: 10)
2. Lesser acne (PSSE contribution: 6)
3. Lower BMI (tending towards normal) (PSSE contribution: 12)

As will be readily apparent to those skilled in the art, the present embodiment may easily be produced in other specific forms without departing from its essential characteristics. The present embodiment are, therefore, to be considered as merely illustrative and not restrictive, the scope being indicated by the claims rather than the foregoing description, and all changes which come within therefore intended to be embraced therein.

We claim:

1. A method for diagnosis and monitoring of poly-cystic ovarian syndrome in a subject by analysing a biological sample taken from said subject, said method comprising detecting one or more of reproductive hormones or their metabolites from the list consisting of E3G, LH, PDG, FSH, DHEA and Testosterone in free or bound form.

2. The method as claimed in claim 1 further comprising:
measuring the levels of said reproductive hormones or their metabolites during at least one menstrual cycle;
determining a maximum concentration of said reproductive hormones or their metabolites during at least one menstrual cycle;
determining simultaneously, for the same cycle, for one or more of said hormones whether the concentration of one or more of said hormones is greater than or lesser than a threshold value during follicular phase of said cycle.

3. A method for diagnosis of poly-cystic ovarian syndrome in a subject by analysing a biological sample taken from said subject, said method comprising:
quantifying concentration/level of luteinizing hormone (LH) and of Estrogen metabolite in the sample during at least one menstrual cycle;
determining a maximum concentration/level LHₘₐₓ of LH and a maximum concentration/level Eₘₐₓ of an Estrogen metabolite during at least one menstrual cycle;
determining, simultaneously, for the same cycle, whether the E3G levels in said sample are more than a first threshold during follicular phase of said cycle and whether the LH levels are less than a second threshold for at least four days during follicular phase of said cycle.

4. The method as claimed in claim 3 wherein the first threshold is 0.8 times Eₘₐₓ and the second threshold 0.095 times LHₘₐₓ.

5. The method as claimed in claim 2 or 3, wherein said follicular phase is at least 4 days between 8^{th} and 13^{th} day.

6. The method as claimed in claim 1 or 3 further comprising:
placing said biological sample over a sample receiving well/zone of a test strip;
reacting the treated biological sample with a detector reagent (a conjugate) containing a detector antibody, which conjugates with said hormones or their metabolites in said biological sample and flows with said biological sample;
receiving of said biological sample fluid along with said detector antibody on at least one test and control lines having immobilized biochemical reagent leading to a coloured reaction giving at least one characteristic colour; and
quantification of said hormones or their metabolites in said biological sample by comparing said characteristic colours against control.

7. The method as claimed in claim 2 or 4 further comprising scanning said immunochromatographic assay strips by a handheld device having an imaging sensor for quantification of said hormones in said sample by comparing the colour on said strips against a standard or control.

8. A method for diagnosing poly-cystic ovarian syndrome (PCOS) in a female subject by analysing a urine sample in said female subject, said method comprising:
measuring levels of luteinizing hormone (LH) and an Estrogen metabolite in a biological sample during follicular phase of a menstrual cycle;
determining whether said Estrogen metabolite levels in said biological sample are more than 40 ng/mL along with whether LH levels are less than 2.4 mIU/mL [0.020 ng/ml] during said follicular phase of said menstrual cycle.

9. The method as claimed in claim 10, further comprising:
placing said urine sample over a sample receiving pad of a test strip, that pre-treats said biological sample;
reacting the treated urine sample with a coloured detector reagent (a conjugate) containing a detector antibody, which is re-hydrated and flows with said urine sample through a membrane of said test strip,
receiving of said urine sample fluid along with said detector antibody on at least one test and control lines having immobilized biochemical reagent leading to a coloured reaction giving at least one characteristic colour; and
quantification of said Estrogen metabolite and LH in said urine sample by comparing said characteristic colours against control.

10. The method as claimed in claim 10, wherein said method is performed by scanning test strip from a handheld device.

11. A handheld computer-implemented system for diagnosing or predicting poly-cystic ovarian syndrome (PCOS) by measuring levels of reproductive hormones or their metabolites in a biological sample of a subject comprising:
a plurality of test strips (202, 204 . . . 20N) to receive said biological sample, wherein said test strips are immunochromatographic assay strips to detect a plurality of reproductive hormones or their metabolites
an imaging sensor (204) to scan said test strips (202, 204, 20N);
a first hormone or metabolites module (206) that is in communication with said imaging sensor (204) and receives scanned images from said imaging sensor (204) to quantify said immunochromatographic assay by comparing the colour on said strips against a standard or control;
a second hormone or metabolite quantification module (208) that is in communication with said imaging sensor (204) and receives scanned images from said imaging sensor (204) to quantify said immunochromatographic assay strips by comparing the colour on said strips against a standard or control;
an analytics module (210) that is in communication with said first quantification module (206) and said second quantification module (208) for plotting quantification data from said quantification module (206) and said quantification module (208); and
a diagnostics module (212) that is in communication with said analytics module (210); wherein said diagnostics module (212) determines and compares hormonal levels and predicts or diagnoses PCOS.

12. The system as claimed in claim 13, wherein said first quantification module is E3G quantification module (206) and said second quantification module is LH quantification module (208).

13. The system as claimed in claim 15, wherein said diagnostics module (212) determines, simultaneously, for same cycle, whether said Estrogen metabolite levels in said sample are more than 0.8 times Eₘₐₓ during follicular phase of said cycle and whether the LH levels are less than 0.095 times LHₘₐₓ during follicular phase of said cycle.

14. The system as claimed in claim 16, wherein said diagnostics module (212) determines whether said Estrogen metabolite levels in a urine sample is more than 17 ng/mL along with LH levels of less than 2.4 mIU/mL [0.020 ng/ml] during follicular phase of said menstrual cycle.

15. The system as claimed in claim 17, wherein said Estrogen metabolite quantification module (206), said LH quantification module (208), said analytics module (210) and said diagnostics module (212) are hosted on a remote server that is communication with said handheld device (106).

16. A method of monitoring PCOS in a subject comprising:
computing a PCOS Severity Score Hormonal (PSSH) (504A), wherein the PSSH is computed on basis of concentration/levels of a plurality of reproductive hormones during a menstrual cycle;
computing a PCOS Severity Score External (PSSE) (504B), wherein the PSSE is computed on basis of the declaration by the subject of the severity of external symptoms of PCOS;
determining (506) an intervention of medication, diet and/or exercise based on the PSSE and the PSSH; and
monitoring the PSSE and PSSH after more than one cycle to determine effect of the intervention.

17. The method as claimed in claim 16, wherein the reproductive hormones are selected from Eesterone-3-glucuronide (E3G), Luteinizing Hormone (LH), Pregnanediol Glucuronide (PDG), Follicle Stimulating Hormone (FSH), Dehydroepiandrosterone (DHEA) and Testosterone in free or bound form

18. The method as claimed in claim 17, wherein the PSSH is computed on basis of FSH levels, LH:FSH ratio, non-ovulatory high LH, absence of coasting type E3G, recurrent oscillation in LH levels, and multiple PdG peaks.

19. The method as claimed in claim 17, wherein the PSSE is computed based on one or more of the following cycle parameters/physical characteristics patterns: frequency of anovulatory cycles, cycle length variation, phenotypic occurrences selected from facial hair, acne, baldness or thinning hair, and weight gain.
